SEQUENCE LISTING (43 sequences)

SEQ ID NO: 1 - protein, 236 aa, Artificial Sequence
  Description of Artificial Sequence Synthetic polypeptide
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 2 - protein, 235 aa, Artificial Sequence
  Description of Artificial Sequence Synthetic polypeptide
MVGEDSVLITENMHMKLYMEGTVNDHHFKCTSEGEGKPYEGTQTMKIKVVEGGPLPFAFDILATSFMYGSKTFINHTQGIPDFFKQSFPEGFTWERITTYEDGGVLTATQDTSLQNGCLIYNVKINGVNFPSNGPVMQKKTLGWEASTEMLYPADSGLRGHSQMALKLVGGGYLHCSLKTTYRSKKPAKNLKMPGFYFVDRRLERIKEADKETYVEQHEMAVARYCDLPSKLGHS

SEQ ID NO: 3 - protein, 234 aa, Artificial Sequence
  Description of Artificial Sequence Synthetic polypeptide
MGEDSELISENMHMKLYMEGTVNGHHFKCTSEGEGKPYEGTQTAKIKVVEGGPLPFAFDILATSFMYGSKTFINHTQGIPDFFKQSFPEGFTWERITTYEDGGVLTATQDTSLQNGCLIYNVKINGVNFPSNGPVMQKKTLGWEASTEMLYPADSGLRGHSQMALKLVGGGYLHCSLKTTYRSKKPAKNLKMPGFYFVDRKLERIKEADKETYVEQHEMAVARYCDLPSKLGHS

SEQ ID NO: 4 - protein, 243 aa, Artificial Sequence
  Description of Artificial Sequence Synthetic polypeptide
MSSGALLFHGKIPYVVEMEGNVDGHTFSIRGKGYGDASVGKVDAQFICTTGDVPVPWSTLVTTLTYGAQCFAKYGPELKDFYKSCMPDGYVQERTITFEGDGNFKTRAEVTFENGSVYNRVKLNGQGFKKDGHVLGKNLEFNFTPHCLYIWGDQANHGLKSAFKICHEITGSKGDFIVADHTQMNTPIGGGPVHVPEYHHMSYHVKLSKDVTDHRDNMSLKETVRAVDCRKTYDFDAGSGDTS

SEQ ID NO: 5 - protein, 215 aa, Artificial Sequence
  Description of Artificial Sequence Synthetic polypeptide
MVSVIKPEMKMRYYMDGSVNGHEFTIEGEGTGRPYEGHQEMTLRVTMAKGGPMPFAFDLVSHVXHRPFTKYPEEIPDYFKQAFPEGLSWERSLEFEDGGSASVSAHISLRGNTFYHKSKFTGVNFPADGPIMQNQSVDWEPSTEKITASDGVLKGDVTMYLKLEGGGNHKCQFKTTYKAAKKILKMPGSHYISHRLVRKTEGNITELVEDAVAHS

SEQ ID NO: 6 - protein, 225 aa, Artificial Sequence
  Description of Artificial Sequence Synthetic polypeptide
MDSTENVIKPFMRFKVHMEGSVNGHEFEIEGEGEGKPYEGTQTAKLQVTKGGPLPFAWDILSPQFQYGSKVYVKHPADIPDYKKLSFPEGFKWERVMNFEDGGVVTVTQDSSLQDGTFIYHVKFIGVNFPSDGPVMQKKTLGWEPSTERLYPRDGVLKGEIHKALKLKGGGHYLVEFKSIYMAKKPVKLPGYYYVDSKLDITSHNEDYTVVEQYERAEARHHLFQ

SEQ ID NO: 7 - protein, 60 aa, Unknown
  Description of Unknown Chloroplast stroma signal polypeptide
MASSMLSSAAVVTSPAQATMVAPFTGLKSSASFPVTRKANNDITSITSNGGRVSCMKVWP

SEQ ID NO: 8 - protein, 77 aa, Unknown
  Description of Unknown Thylakoid membrane signal polypeptide
MASMGGLHGASPAVLEGSLKINGSSRLNGSGRVAVAQRSRLVVRAQQSEETSRRSVIGLVAAGLAGGSFVQAVLADA

SEQ ID NO: 9 - protein, 84 aa, Unknown
  Description of Unknown Thylakoid membrane signal polypeptide
MAQAVTSMAGLRGASQAVLEGSLQINGSNRLNISRVSVGSQRTGLVIRAQQNVSVPESSRRSVIGLVAAGLAGGSFVKAVFAEA

SEQ ID NO: 10 - protein, 83 aa, Spinacia oleracea
MAQAMASMAGLRGASQAVLEGSLQISGSNRLSGPTTSRVAVPKMGLNIRAQQVSAEAETSRRAMLGFVAAGLASGSFVKAVLA

SEQ ID NO: 11 - protein, 82 aa, Onobrychis viciifolia
MAQAMASGLLEGSLQLMSGSNRSSSSSRTRGPAGVFIVRAQQQEQQQGMSSSAADPQSNRRAMLGLVATGLASASFVQAVLA

SEQ ID NO: 12 - protein, 90 aa, Arabidopsis thaliana
MAFSSLSPLPMKSLDISRSSSSVSRSPYHFQRYLLRRLQLSSRSNLEIKDSSNTREGCCSSAESNTWKRILSAAMAAAVIASSSGVPAMA

SEQ ID NO: 13 - protein, 98 aa, Arabidopsis thaliana
MWSQSFLGSAPKLCLFSSSLPPFSHHKIHKFFCFAQNPSSTVSINLSKRHLNLSILTLFFNGFLLDNKAKSMEELQRYTDSNNGFTLLIPSSYTKVEK

SEQ ID NO: 14 - protein, 84 aa, Arabidopsis thaliana
METLLSPRALSPPLNPKPLSLHQTKPTSHSLSLSKPTTFSGPKHLSTRFTKPESRNWLIDAKQGLAALALSLTLTFSPVGTALA

SEQ ID NO: 15 - protein, 78 aa, Arabidopsis thaliana
MSVSLSAASHLLCSSTRVSLSPAVTSSSSSPVVALSSSTSPHSLGSVASSSLFPHSSFVLQKKHPINGTSTRMISPKC

SEQ ID NO: 16 - protein, 92 aa, Arabidopsis thaliana
MAAAFASLPTFSVVNSSRFPRRRIGFSCSKKPLEVRCSSGNTRYTKQRGAFTSLKECAISLALSVGLMVSVPSIALPPNAHAVANPVIPDVS

SEQ ID NO: 17 - protein, 114 aa, Arabidopsis thaliana
MASPLSSSTVVSHRLFFLHPSPLNRKFLFVKPKLPFNRTNSGDFRMRLHSTSSKTGTKELIHSCNSSIDSKLNTFEAGSKNLEKLVATILIFVQVWSPLPLFGLDSAYISPAEA

SEQ ID NO: 18 - protein, 75 aa, Arabidopsis thaliana
MEALQFSSVNRVPCTLSCTGNRRIKAAFSSAFTGGTINSASLSSSRNLSTREIWSWVKSKTVVGHGRYRRSQVRA

SEQ ID NO: 19 - protein, 92 aa, Arabidopsis thaliana
MASSAAQIHILGGIGFPTSSSSSSTKNLDNKTNSIPRSVFFGNRTSPFTTPTSAFLRMGRRNNNASRYTVGPVRVVNEKVVGIDLGTTNSAV

SEQ ID NO: 20 - protein, 92 aa, Arabidopsis thaliana
MASSAAQIHVLGGIGFASSSSSKRNLNGKGGTFMPRSAFFGTRTGPFSTPTSAFLRMGTRNGGGASRYAVGPVRVVNEKVVGIDLGTTNSAV

SEQ ID NO: 21 - protein, 236 aa, Artificial Sequence
  Description of Artificial Sequence Synthetic polypeptide
MVSKGEENNMAIIKEFMRFKVRMEGSVNGHEFEIEGEGEGRPYEGFQTAKLKVTKGGPLPFAWDILSPQFTYGSKAYVKHPADIPDYFKLSFPEGFKWERVMNFEDGGVVTVTQDSSLQDGEFIYKVKLRGTNFPSDGPVMQKKTMGWEASSERMYPEDGALKGEIKMRLKLKDGGHYTSEVKTTYKAKKPVQLPGAYIVGIKLDITSHNEDYTIVEQYERAEGRHSTGGMDELYK

SEQ ID NO: 22 - protein, 236 aa, Artificial Sequence
  Description of Artificial Sequence Synthetic polypeptide
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGSMDELYK

SEQ ID NO: 23 - protein, 231 aa, Artificial Sequence
  Description of Artificial Sequence Synthetic polypeptide
MSELIKENMHMKLYMEGTVNNHHFKCTSEGEGKPYEGTQTMKIKVVEGGPLPFAFDILATSFMYGSKAFINHTQGIPDFFKQSFPEGFTWERITTYEDGGVLTATQDTSFQNGCIIYNVKINGVNFPSNGPVMQKKTRGWEANTEMLYPADGGLRGHSQMALKLVGGGYLHCSFKTTYRSKKPAKNLKMPGFHFVDHRLERIKEADKETYVEQHEMAVAKYCDLPSKLGHR

SEQ ID NO: 24 - protein, 232 aa, Artificial Sequence
  Description of Artificial Sequence Synthetic polypeptide
MSSGALLFHGKIPYVVEMEGNVDGHTFSIRGKGYGDASVGKVDAQFICTTGDVPVPWSTLVTTLTYGAQCFAKYGPELKDFYKSCMPDGYVQERTITFEGDGNFKTRAEVTFENGSVYNRVKLNGQGFKKDGHVLGKNLEFNFTPHCLYIWGDQANHGLKSAFKICHEITGSKGDFIVADHTQMNTPIGGGPVHVPEYHHMSYHVKLSKDVTDHRDNMSLKETVRAVDCRKT

SEQ ID NO: 25 - protein, 279 aa, Artificial Sequence
  Description of Artificial Sequence Synthetic polypeptide
MSSGALLFHGKIPYVVEMEGDVDGHTFSIRGKGYGDASVGKVDAQFICTTGDVPVPWSTLVTTLTYGAQCFAKYGPELKDFYKSCMPDGYVQERTITFEGDGNFKTRAEVTFENGSVYNRVKLNGQGFKKDGHVLGKNLEFNFTPHCLYIWGDQANHGLKSAFKICHEIAGSKGDFIVADHTQMNTPIGGGPVHVPEYHHMSYHVKLSKDVTDHRDNMSLTETVRAVDCRKTYLGSRDISHGFPPAVAAQDDGTLPMSCAQESGMDRHPAACASARINV

SEQ ID NO: 26 - protein, 214 aa, Artificial Sequence
  Description of Artificial Sequence Synthetic polypeptide
MEGSVNGHEFEIEGEGEGRPYEGFQTAKLKVTKGGPLPFAWDILSPHFTYGSKAYVKHPADIPDYFKLSFPEGFKWERVMNYEDGGVVTVTQDSSLQDGEFIYKVKLRGTNFPSDGPVMQKKTMGWEASSERMYPEDGALKGKIKMRLKLKDGGHYTSEVKTTYKAKKPVQLPGAYIVDIKLDITSHNEDYTIVEQYERAEGRHSTGGMDELYK

SEQ ID NO: 27 - protein, 218 aa, Artificial Sequence
  Description of Artificial Sequence Synthetic polypeptide
MVSVIKPEMKMKYFMDGSVNGHEFTVEGEGTGKPYEGHQEMTLRVTMAKGGPMPFSFDLVSHTFCYGHRPFTKYPEEIPDYFKQAFPEGLSWERSLQFEDGGFAAVSAHISLRGNCFEHKSKFVGVNFPADGPVMQNQSSDWEPSTEKITTCDGVLKGDVTMYLKLAGGGNHKCQFKTTYKAAKKILKMPQSHFIGHRLVRKTEGNITELVEDAVAHC

SEQ ID NO: 28 - protein, 214 aa, Artificial Sequence
  Description of Artificial Sequence Synthetic polypeptide
MRFKVRMEGTVNGHEFEIEGEGEGRPYEGHNTVKLKVTKGGPLPFAWDILSPQFQYGSKVYVKHPADIPDYKKLSFPEGFKWERVMNFEDGGVATVTQDSSLQDGCFIYKVKFIGVNFPSDGPVMQKKTMGWEASTERLYPRDGVLKGETHKALKLKDGGHYLVEFKSIYMAKKPVQLPGYYYVDAKLDITSHNEDYTIVEQYERTEGRHHLFL

SEQ ID NO: 29 - protein, 79 aa, Artificial Sequence
  Description of Artificial Sequence Synthetic polypeptide
MVSKGEELFTGVVPILVELDGDVNGHKFSVRGEGEGDATNGKLTLKFICTTGKLPVPWPTLVTTLSHGVQCFARYPDHM

SEQ ID NO: 30 - protein, 219 aa, Artificial Sequence
  Description of Artificial Sequence Synthetic polypeptide
MEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 31 - protein, 265 aa, Artificial Sequence
  Description of Artificial Sequence Synthetic polypeptide
MVSKGEELFTGVVPILVELDGDVNGHKFSVSGEGEGDATYGKLTLKFICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKATLLYIVDNSLAVVLQRRDWENDLTD

SEQ ID NO: 32 - protein, 476 aa, Artificial Sequence
  Description of Artificial Sequence Synthetic polypeptide
MVSKGEEVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLGHGTGSTGSGSSGTASSEDNNMAVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLYGMDELYK

SEQ ID NO: 33 - protein, 227 aa, Artificial Sequence
  Description of Artificial Sequence Synthetic polypeptide
MAIIKEFMRFKVRMEGSVNGHEFEIEGEGEGRPYEGTQTAKLKVTKGGPLPFAWDILTPNFTYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKMRLKLKDGGHYDAEVKTTYKAKKPVQLPGAYIVGIKLDITSHNEDYTIVELYERAEGRHSTGGMDELYK

SEQ ID NO: 34 - protein, 235 aa, Artificial Sequence
  Description of Artificial Sequence Synthetic polypeptide
MVGEDSELITENMHMKLYMEGTVNNHHFKCTSEGEGKPYEGTQTMKIKVVEGGPLPFAFDILATSFMYGSKAFINHTQGIPDFFKQSFPEGFTWERITTYEDGGVLTATQDTSLQNGCLIYNVKINGVNFPSNGPVMQKKTLGWEASTEMLYPADSGLRGHGQMALKLVGGGYLHCSLKTTYRSKKPAKNLKMPGFHFVDHRLERIKEADKETYVEQHEMAVAKYCDLPSKLGHS

SEQ ID NO: 35 - protein, 79 aa, Artificial Sequence
  Description of Artificial Sequence Synthetic polypeptide
MVSELIKENMHMKLYMEGTVNNHHFKCTSEGEGKPYEGTQTMRIKAVEGGPLPFAFDILATSFMYGSKTFINHTQGIPD

SEQ ID NO: 36 - protein, 225 aa, Artificial Sequence
  Description of Artificial Sequence Synthetic polypeptide
MDSTENVIKPFMRFKVHMEGSVNGHEFEIEGVGEGKPYEGTQTAKLQVTKGGPLPFAWDILSPQFFYGSKAYIKHPADIPDYLKQSFPEGFKWERVMNFEDGGVVTVTQDSSLQDGTLIYHVKFIGVNFPSDGPVMQKKTLGWEPSTERNYPRDGVLKGENHMALKLKGGGHYLCEFKSIYMAKKPVKLPGYHYVDYKLDITSHNEDYTVVEQYERAEARHHLFQ

SEQ ID NO: 37 - protein, 238 aa, Artificial Sequence
  Description of Artificial Sequence Synthetic polypeptide
MSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 38 - protein, 237 aa, Artificial Sequence
  Description of Artificial Sequence Synthetic polypeptide
MVSKGEELIKENMHMKLYMEGTVNNHHFKCTSEGEGKPYEGTQTMRIKVVEGGPLPFAFDILATSFMYGSRTFINHTQGIPDFFKQSFPEGFTWERVTTYEDGGVLTATQDTSLQDGCLIYNVKIRGVNFPSNGPVMQKKTLGWEANTEMLYPADGGLEGRSDMALKLVGGGHLICNFKTTYRSKKPAKNLKMPGVYYVDHRLERIKEADKETYVEQHEVAVARYCDLPSKLGHKLN

SEQ ID NO: 39 - protein, 244 aa, Artificial Sequence
  Description of Artificial Sequence Synthetic polypeptide
MSKGEELFTGVVPILVELDGDVNGHKFSVSGEGEGDATYGKLTLKFICTTGKLPVPWPTLVTTLSHGVQCFSRYPDHMKQHDFFKSAMPEGYVQERTIFFKDDGNYKTRAEVKFEGDTLVNRIELKGIDFKEDGNILGHKLEYNFNSHNIYIMADKQKNGIKVNFKIRHNIEDGSVQLADHYQQNTPIGDGPVLLPDNHYLSTQSALSKDPNEKRDHMVLLEFRTAAGITHGMDELYKHHHHHH

SEQ ID NO: 40 - protein, 237 aa, Artificial Sequence
  Description of Artificial Sequence Synthetic polypeptide
MVSKGEELFTGVVPILVELDGDVNGHKFSVSGEGEGDATYGKLTLKLICTTGKLPVPWPTLVTTLXLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALSKDPNEKRDHMVLLEFVTAAGITHGMDELYK

SEQ ID NO: 41 - protein, 80 aa, Artificial Sequence
  Description of Artificial Sequence Synthetic polypeptide
MVSKGEELFTGVVPILVELDGDVNGHKFSVSGEGEGDATYGKLTLKLICTTGKLPVPWPTLVTTLGYGLQCFARYPDHMK

SEQ ID NO: 42 - protein, 215 aa, Artificial Sequence; identical to SEQ ID NO: 5
  Description of Artificial Sequence Synthetic polypeptide

SEQ ID NO: 43 - protein, 83 aa, Artificial Sequence
  Description of Artificial Sequence Synthetic polypeptide
MSRRRHSYENDGGQPHKRRKTFPPVATMSELIKENMHMKLYMEGTVNNHHFKCTSEGEGKPYEGTQTMRIKVVEGGPLPFAFD

(19) United States
(12) Patent Application Publication
(10) Pub. No.: US 2013/0333073 A1
(43) Pub. Date: Dec. 12, 2013

(54) Compositions and Methods for Enhancing Plant Photosynthetic Activity

(71) Applicant: Futuragene Israel Ltd., Park Tamar (IL)

(72) Inventors: Daniel Siegel, Rehovot (IL); Dror Avisar, Kochav Yair (IL); Miron Abramson, Karmei Yosef (IL); Ziv Shani, Mazkeret Batia (IL); Stanley Hirsch, Pardesiya (IL)

(21) Appl. No.: 13/912,635

(22) Filed: Jun. 7, 2013

Related U.S. Application Data
(60) Provisional application No. 61/656,794, filed on Jun. 7, 2012.
(60) Provisional application No. 61/672,500, filed on Jul. 17, 2012.

(51) Int. Cl. A01H 5/10; A01C 14/00; A01G 1/00

(52) U.S. Cl. CPC A01H 5/10; A01G 1/001; A01C 14/00
USPC 800/298; 47/58.1 R; 111/200

(57) ABSTRACT

Methods for improving the efficiency of photosynthesis in plants exposed to suboptimal light conditions. Photosynthesis enhancement is achieved by transformation and expression of one or more exogenous chromophores in the chloroplast of plants or in the cytoplasm under the control of a transit peptide which directs it to the chloroplast or a compartment within the chloroplast. Preferred chromophores have excitation max in the green-yellow light spectrum. Chains of chromophores can be used to capture and emit light from one to the other until the emitted wave length is in the range that can be efficiently utilize by the native light harvest complex.

### CROSS REFERENCE TO RELATED APPLICATIONS

This application claims priority under 35 U.S.C. 119(e) of provisional applications 61/656,794, filed Jun. 7, 2012 and 61/672,500, filed Jul. 17, 2012. Each of the foregoing provisional applications is hereby incorporated by reference in its entirety.

### SEQUENCE LISTING

The instant application contains a Sequence Listing which has been submitted in ASCII format via EFS-Web and is hereby incorporated by reference in its entirety. Said ASCII copy, created on Jun. 7, 2013, is named 30407-0005001_SL.txt and is 70,785 bytes in size.

### TECHNICAL FIELD

This invention relates to transgenic plants with enhanced photosynthetic capabilities, and more particularly to such plants with enhanced photosynthetic ability under low light conditions.

### BACKGROUND

Photosynthetic plants depend on light, e.g., sunlight, as their energy source. It is generally accepted that light capture and the light reactions of photosynthesis are typically not limiting to plant productivity in agricultural settings. Such acceptance, however, stems from typical academic greenhouse studies and is not generally correct. Plants often encounter light conditions that are suboptimal for growth. The dawn and late afternoon hours, for example, are characterized as having lower light intensity and correspondingly lower photosynthesis rates. The diurnal changes of photosynthesis rate are affected by the photosynthetic photon flux density (PPFD). Plants also often compete for sunlight. Taller-growing plants frequently configure a canopy that absorbs light and influences photosynthetic and growth rates of lower-growing plants that do not reach to the canopy. Leaves that are shaded by other leaves have much lower photosynthetic rates. Shading is also observed upon high density planting of row crops.

Photosynthetic activity may also be constrained by a plant's inability to efficiently utilize the full spectrum of light. The light that drives the photochemical reactions of photosynthesis is first absorbed by the plant chloroplast pigments. The chlorophylls are the typical pigments of photosynthetic organisms. Chlorophyll a has two peaks of optimal efficiency, one in the blue part of the spectrum (around 430 nm) and one in the red part of the spectrum (680 nm). Various “associated pigments” absorb light in other parts of the visible spectrum, with most of the energy absorbed being passed through a chain of receptors until the energy is equivalent to that absorbed at 700 nm. Photosynthesis, however, is not driven effectively by light in the green-yellow part of the spectrum, i.e., by light having wavelengths in the range of 520-550 nm. Light reaching lower leaves in dense forestry stands is very green, making such light further suboptimal for driving photosynthesis. Part of the light spectrum is thus unavailable to drive photosynthesis or drives photosynthesis inefficiently.

Plant photosynthetic activity may thus be limited by light intensity and the limited spectrum of light wavelengths at which endogenous pigments of plants are optimized to absorb light and drive the photosynthetic process. The present invention provides for methods and compositions for improving photosynthetic activity of plants, by providing plants with non-endogenous chromophores that absorb and emit light (i.e., photophores) that enable plants to more efficiently utilize a broader band of light wavelengths to drive the photosynthetic process. The invention provides for improved plant growth under suboptimal conditions, such as under low light conditions.

### SUMMARY

The present invention is directed to compositions and methods for improving growth of plants.

In some aspects, a transgenic plant with improved photosynthetic activity is provided, where the plant contains an exogenous chromophore that absorbs a first wavelength of light in a range that is suboptimal for photosynthetic activity and which, upon absorbing such a wavelength of light, emits light at a wavelength in a range that is effective for photosynthetic activity in the plant.

In some aspects, a transgenic plant with improved photosynthetic activity is provided, where the plant includes a chain of chromophores that absorbs a wavelength of light in a range that is suboptimal for photosynthetic activity and which then passes energy through the chain until a chromophore emits light at a wavelength in a range that is effective for photosynthetic activity in the plant. For example, a transgenic plant can include a second exogenous chromophore that absorbs a wavelength of light in a range that is suboptimal for photosynthetic activity in the plant and upon absorbing the wavelength of light emits the first wavelength of light in a range that is suboptimal for photosynthetic activity and which is absorbed by the first exogenous chromophore, which then emits a wavelength of light that is effective for photosynthetic activity in the plant. In some embodiments, the second exogenous chromophore has a maximum emission wavelength that is identical or near the maximum absorption wavelength of the first chromophore.

In some aspects, the exogenous chromophores described above are localized to the chloroplast of the plant, preferably to the thylakoid membrane of the plant.

In some aspects, a method of co-cultivating plants is provided, wherein a transgenic plant with improved photosynthetic capabilities is co-cultivated with another plant under conditions that shade the transgenic plant.

In some aspects, a method of increasing the nitrogen content of soil is provided, the methods including growing a transgenic nitrogen-fixing plant, e.g., a legume, expressing an exogenous chromophore in soil.

### BRIEF DESCRIPTION OF THE DRAWINGS

FIG. 1 is a schematic representation of a chromophore chain that can be used to harvest light in the green wavelength spectrum that is poorly utilized by wild type plants and, through a cascade of energy transfers, convert light to light in the red wavelength spectrum that is efficiently utilized by the light harvesting mechanisms in wild type plants.

FIG. 2 is a set of fluorescent stereoscopic images of wild type and mCherry transgenic eucalyptus leaves (excitation filter BP530-550, barrier filter BA575IF). The mCherry transgenic eucalyptus leaves show significantly greater fluorescence intensity than the wild type eucalyptus leaves.

FIG. 3 is a set of photographs of wild type and mCherry (line 11) transgenic eucalyptus plants after 36 days of growth.

FIG. 4 is a graph showing the average height of wild type and transgenic mCherry plants (lines 9 and 11), from the bottom of the stem to the top, after 36 days of growth. The data shown are the mean±1.96 standard error of at least 37 replicates (*, p<0.05; ANOVA followed by Dunnett's method).

### DETAILED DESCRIPTION

Photosynthetic plants depend on sunlight as their energy source. Thus, they need to detect the intensity, quality, duration and direction of this critical environmental factor and to respond properly by optimizing their growth and development.

Chlorophylls a and b are abundant in green plants. The chlorophylls have a complex ring structure that is chemically related to the porphyrin-like groups found in hemoglobin and cytochromes. Carotenoids are linear molecules with multiple conjugated double bonds that absorb light in the 400 to 500 nm region, giving carotenoids their characteristic orange color. The majority of pigments absorb certain wavelengths of light and reflect non-absorbed wavelengths and function as parts of antenna complexes, collecting light and transferring the absorbed energy to the chlorophylls in the reaction center complex, where the chemical oxidation and reduction reactions leading to long-term energy storage take place.

Antenna systems function to deliver energy efficiently to the reaction centers with which they are associated. The molecular structures of antenna pigments are quite diverse, although all of them are associated in some way with the photosynthetic membrane. The physical mechanism by which excitation energy is conveyed from the chlorophyll that absorbs the light to the reaction center is thought to be resonance transfer (Resonance Energy Transfer-RET). By this mechanism the excitation energy is transferred from one molecule to another by a non-radiative process. Light absorbed by carotenoids or chlorophyll b in the light harvest complex proteins is rapidly transferred to chlorophyll a and then to other antenna pigments that are intimately associated with the reaction center.

Under sub-optimal light conditions the plants could benefit from enhanced light utilization spectrum, compared to wild type plants, by absorption of wavelengths that wild type plants are not adapted to absorb (e.g., 520-640 nm). The transgenic plants described herein have enhanced photosynthesis capabilities due the capture of more photons from light wavelengths that are suboptimal for photosynthetic activity in wild type plants. The utilization of such suboptimal wavelengths allows the transgenic plants to generate energy for increased photosynthetic rates compared to wild type plants, thus increasing biomass accumulation and growth.

Chromophores and Energy Transfer in the Chloroplast

Transgenic plants benefit from enhanced light utilization spectrum, compared to wild type plants, by absorption of wavelengths that wild type plants are not adapted to absorb or utilize efficiently. Preferred chromophores have excitation max in the green-yellow light spectrum (approximately 520-550 nm) that is inefficient in driving photosynthesis in green plants. To drive photosynthetic activity, chromophore(s) should singly or in combination generate energy that can be captured and utilized by the native light harvesting complex. Chains of chromophores can be used to capture and emit light from one to the other until the emitted wave length is in the range that can be efficiently utilized by the native light harvest complex.

Different pigments together serve as an antenna, collecting light and transferring its energy to the reaction center. Antenna systems function to deliver energy efficiently to the reaction centers with which they are associated. (van Grondelle et al., Biochem. Biophys. ACTA, 1187:1-65, 1994; Pullerits and Sundström, Ace Chem Res, 29:381-389, 1996). The molecular structures of antenna pigments are quite diverse, although all of them are associated in some way with the photosynthetic membrane. The physical mechanism by which excitation energy is conveyed from the chlorophyll that absorbs the light to the reaction center is thought to be resonance transfer (Resonance Energy Transfer—RET). By this mechanism the excitation energy is transferred from one molecule to another by a non-radiative process.

Photosynthetic efficiency can be increased by overexpressing endogenous chromophores or expressing exogenous chromophores. Endogenous chromophores include the chlorophylls and carotenoids. Chlorophyll a has two peaks of optimal efficiency, one in the blue part of the spectrum (around 430 nm) and one in the red part of the spectrum (680 nm), there are “associated pigments” which take advantage of nearly every part of the visible spectrum, and most of the energy absorbed is passed along a chain of receptors (losing bits along the way, of course) until the energy is equivalent to that absorbed at 700 nm. Carotenoids are linear conformation molecules with multiple conjugated double bonds. Absorption bands in the 400 to 500 nm region give carotenoids their characteristic orange color. The majority of the pigments serve as an antenna complex, collecting light and transferring the energy to the reaction center complex, where the chemical oxidation and reduction reactions leading to long-term energy storage take place. Light absorbed by carotenoids or chlorophyll b in the light harvest complex proteins is rapidly transferred to chlorophyll a and then to other antenna pigments that are intimately associated with the reaction center.

Photosynthetic efficiency may also be enhanced by expressing exogenous fluorescent proteins that absorb light at wavelengths that are photosynthetically poorly-utilized by the native plant systems. Light wavelengths in the green-yellow spectrum (520-590 nm), for example, are poorly absorbed by the native light harvesting complexes. Transgenic proteins preferably emit light in the native photosynthetic range of the recipient organism by means of resonance energy transfer (RET) and thus participate in energy transfer within the plant. The RET process includes excitation of a first transgenic chromophore molecule which in turn transfers its energy by emission at a wavelength that can be absorbed by a second chromophore adapted to absorb energy at the emission spectrum of the first chromophore. The process of energy transfer from one chromophore to another may take place via emission and excitation or photon transfer or any other means of energy transfer between two chromophores or light harvest elements.

Examples of exogenous chromophores that may be used to enhance photosynthetic activity of plants are shown in Table 1.

Non-limiting examples of chromophores that can be used to enhance photosynthetic activity are given in Table 1. Additional exemplary chromophores include mVenus (SEQ ID NO: 41), mFred (SEQ ID NO: 42) and mKate1 (SEQ ID NO: 43).

TABLE 1
Fluorescent proteins data

| | | | | | SEQ | |
|---|---|---|---|---|---|---|
| | Excitation | Emission | Quantum | Bright- | ID | |
| Protein | (Max) | (Max) | yield | ness | NO: | Ref. |
| Azurite | 383 | 447 | 0.55 | 14 | 39 | 3 |
| EBFP2 | 383 | 448 | 0.56 | 18 | 29 | |
| Wild Type | 396, 475 | 508 | 0.77 | 16 | 37 | |
| GFP | | | | | | |
| T-Sapphire | 399 | 511 | 0.60 | 26 | | 4 |
| TagBFP | 402 | 457 | 0.63 | 33 | 30 | |
| Topaz | 514 | 527 | 0.60 | 57 | | 5 |
| Venus | 515 | 528 | 0.57 | 53 | 40 | 1, 2, 6 |
| mCitrine | 516 | 529 | 0.76 | 59 | 31 | |
| YPet | 517 | 530 | 0.77 | 80 | | 7 |
| PhiYFP | 525 | 537 | 0.40 | 52 | 24 | |
| PhiYFP-m | 525 | 537 | 0.39 | 48 | 25 | |
| TurboYFP | 525 | 538 | 0.53 | 56 | 4 | |
| Kusabira- | 548 | 559 | 0.60 | 31 | 27 | |
| Orange | | | | | | |
| mKO1 | 548 | 559 | 0.60 | 31 | 5 | |
| mOrange | 548 | 562 | 0.69 | 49 | 21 | |
| mOrange2 | 549 | 565 | 0.60 | 35 | 26 | |
| TurboRFP | 553 | 574 | 0.67 | 62 | 23 | |
| DsRed- | 554 | 591 | 0.42 | 15 | 6 | |
| Express2 | | | | | | |
| tdTomato | 554 | 581 | 0.69 | 95 | 32 | |
| TagRFP | 555 | 584 | 0.48 | 48 | 38 | |
| DsRed2 | 563 | 582 | 0.55 | 24 | 28 | |
| (“RFP”) | | | | | | |
| mStrawberry | 574 | 596 | 0.29 | 26 | 33 | |
| TurboFP602 | 574 | 602 | 0.35 | 26 | 34 | |
| mCherry | 587 | 610 | 0.22 | 16 | 1, 22 | |
| mKate | 588 | 635 | 0.30 | 15 | | 8 |
| (TagFP635) | | | | | | |
| mKate2 | 588 | 633 | 0.40 | 25 | 35 | |
| E2-Crimson | 611 | 646 | 0.23 | 29 | 36 | |

¹David et al., Photochem. Photobiol. Sci. 11: 358-363, 2012.
²Sarker et al., J. Biomed. Opt. 14:34-37, 2009.
³Mena et al., Nat. Biotechnol. 24:1569-1571, 2006.
⁴Zapata-Hommer et al., BMC Biotechnol. 3:5, 2003.
⁵Han et al., Ann. N.Y. Acad. Sci. 971:627-633, 2002.
⁶Nagai et al., Nat. Biotechnol. 20:87-90, 2002.
⁷Shimozono et al., Methods Cell Biol. 85:381-393, 2008.
⁸Pletnev et al., J. Biol. Chem. 283:28980-28987, 2008.

Preferred chromophores are identified as having one or more of the following properties: (i) excitation wavelength range not efficiently utilized by plant, (ii) emission wavelength in a range that can excite a chromophore in an energy transfer chain; (iii) high quantum yield (the ratio between photons emitted and photon absorbed—a number that is 0<n<1), and (iv) high level of brightness, i.e., the intensity of the emission, defined as: Molar Extinction Coefficient×Fluorescence Quantum Yield/1000.

Enhancement of photosynthetic activity may be accomplished by expression of a single transgenic chromophore or two or more chromophores with overlapping emission and absorption spectra. A chain of several chromophores with overlapping emission and excitation spectrum can be used to overcome large gaps between the emission spectrum of one chromophore and the excitation spectrum of another chromophore and/or the acceptor native light harvest pigments and chlorophylls. The second chromophore may be a transgenic chromophore or one or more of the native pigments and/or chlorophylls such as one that is part of the native light harvesting complex. These genes can be expressed in tandem with other genes or used in co-transformations. Two or more fluorescent proteins can be introduced into the cells in order to reach optimal photosynthetic efficiency. The acceptor may be but is not limited to carotenoid or other tetraterpenoid organic pigments, xanthophylls or carotenes or chlorophyll a or b.

An example of pairs and chains of chromophores suitable for use in a chain of chromophores include TurboYFP (excitation max at 525 nm; emission max of 538 nm) and mKO1 (excitation max at 548 nm; emission max of 559 nm. mKO1 in turn is capable of exciting a third chromophore, for example DsRed-Express2 (excitation max at 554 nm, emission max at 591 nm). DsRed-Express2 may also be used to transfer energy to and excite mCherry, TurboFP635, TurboFP650, which in turn emit at the red wavelengths (610-650 nm) that can be utilized by the plant light harvesting complex, including chlorophylls and carotenoids. Co-expression of one or more chromophores with overlapping emission and excitation spectra can be thus used as an artificial chain to capture light in green-yellow spectrum and transfer its energy to the plant light harvesting complex. These exemplary chromophore chains above enable plants to better utilize light in the green-yellow wavelength spectrum between 530-590 nm, thereby enhancing photosynthesis.

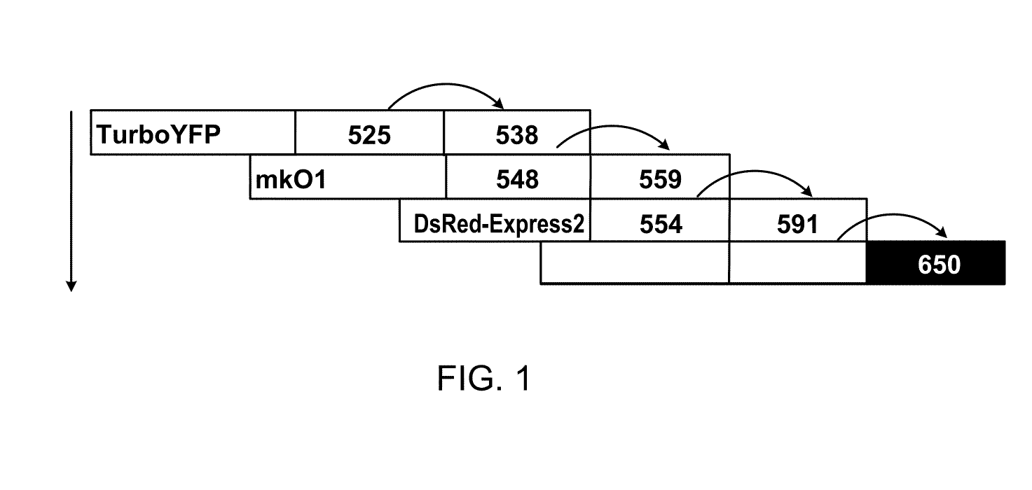

FIG. 1 is a schematic representation of a chromophore chain that can be used to harvest light in the green wavelength spectrum that is poorly utilized by wild type plants and, through a cascade of energy transfers, convert light to light in the red wavelength spectrum that is efficiently utilized by the light harvesting mechanisms in wild type plants.

Photosynthetic Cell Types

Transgenic plants may benefit from enhanced photosynthetic activity in any tissue or cell type that contributes to the photosynthetic activity of the plant. The most active photosynthetic tissue in higher plants is the mesophyll of leaves. Mesophyll cells have multiple copies of chloroplasts, which contain the specialized light-absorbing green pigments, the chlorophylls.

Localization of Exogenous Chromophores

Photosynthesis enhancement is achieved by transformation and expression of one or more exogenous chromophores in the chloroplast of plants and/or in the cytoplasm and/or in the cytoplasm under the control of a transit peptide which directs it to the chloroplast or a compartment within the chloroplast. The thylakoid reactions of photosynthesis take place in the specialized internal membranes of the chloroplast called thylakoids. The end products of these thylakoid reactions are the high-energy compounds ATP and NADPH, which are used for the synthesis of sugars in the carbon fixation reactions which comprise most of the plant (and earth) biomass. The synthesis of sugars takes place in the stroma of the chloroplasts, the aqueous region that surrounds the thylakoids.

Thus, co-expression, for example of SEQ ID NO: 2 or 3 together with SEQ ID NO: 4-6 or SEQ ID NO: 5-6 fused to any chloroplast, stroma or thylakoid signal peptide. Alternatively, plastid transformation vectors carrying the DNA sequence of the chromophores can be used for chloroplast transformation and expression of the genes of interest directly in the chloroplast.

In one embodiment independent expression of SEQ ID NO: 1-3 fused to either chloroplast stroma signal (SEQ ID NO: 7) or more preferably to a thylakoid membrane signal (SEQ ID NO: 8 and 9). Expression of transgenic chromophores in the chloroplast will bring them into close physical proximity with the native light harvesting complex antenna i.e. carotenoid-chlorophyll light harvesting antenna. The RET phenomena occurs most efficiently at distances of up to 10 nm between each chromophore. Therefore optimal energy transfer between transgenic chromophores and the native light harvesting complex occurs when the transgenic chromophores are present in the chloroplast and preferably in the stroma, most preferably in the thylakoids.

Examples of signal peptides that may be used to direct proteins to the thylakoid membrane are provided in SEQ ID NO: 8-17.

Examples of peptides that serve as stromal localization signals are provided in SEQ ID NO: 7 and 18-20.

Expression Constructs and Vectors

Transgenic plant cells and transgenic plants can be generating using a DNA construct or a DNA vector containing a nucleic acid sequence encoding an exogenous chromophore and a promoter operably linked to the nucleic acid sequence encoding the exogenous chromophore. In some embodiments, the DNA construct or vector can further include one or more (e.g., two, three, or four) additional regulatory elements, such as a 5′ leader and/or intron for enhancing transcription, a 3′-untranslated region (e.g., a sequence containing a polyadenylation signal), and a nucleic acid sequence encoding a transit or signal peptide (e.g., a chloroplast transit or signaling peptide)

The choice of promoter(s) that can be used depends upon several factors, including, but not limited to, efficiency, selectability, inducibility, desired expression level, and/or preferential cell or tissue expression. It is a routine matter for one of skill in the art to modulate the expression of a sequence by appropriately selecting and positioning promoters and other regulatory regions relative to that sequence. Examples of promoters that can be used are known in the art. Some suitable promoters initiate transcription only, or predominantly, in certain cell types. Methods for identifying and characterizing promoter regions in plant genomic DNA include, for example, those described in Jordano, et al., Plant Cell 1:855-866, 1989; Bustos, et al., Plant Cell 1:839-854, 1989; Green, et al., EMBO J. 7:4035-4044, 1988; Meier et al., Plant Cell 3:309-316, 1991; and Zhang et al., Plant Physiology 110: 1069-1079, 1996.

Promoters that can be used include those present in plant genomes, as well as promoters from other sources. Exemplary promotes include nopaline synthase (NOS) and octopine synthase (OCS) promoters carried on tumor-inducing plasmids of Agrobacterium tumefaciens and CaMV35S promoters from the cauliflower mosaic virus, see, e.g., the promoters described in U.S. Pat. Nos. 5,164,316 and 5,322,938 (herein incorporated by reference). Non-limiting exemplary promoters derived from plant genes are described in U.S. Pat. No. 5,641,876, which describes a rice actin promoter, U.S. Pat. No. 7,151,204, which describes a maize chloroplast aldolase promoter and a maize aldolase (FDA) promoter, and U.S. Patent Application Publication No. 2003/0131377, which describes a maize nicotianamine synthase promoter (each of which is incorporated herein by reference).

Additional examples of promoters that can be used include ribulose-1,5-bisphosphate carboxylase (RbcS) promoters, such as the RbcS promoter from Eastern larch (Larix laricina), the pine cab6 promoter (Yamamoto et al., Plant Cell Physiol. 35:773-778, 1994), the Cab-1 gene promoter from wheat (Fejes et al., Plant Mol. Biol. 15:921-932, 1990), the CAB-1 promoter from spinach (Lubberstedt et al., Plant Physiol. 104:997-1006, 1994), the cab1R promoter from rice (Luan et al., Plant Cell 4:971-981, 1992), the pyruvate orthophosphate dikinase (PPDK) promoter from maize (Matsuoka et al., Proc. Natl. Acad. Sci. U.S.A. 90:9586-9590, 1993), the tobacco Lhcb1*2 promoter (Cerdan et al., Plant Mol. Biol. 33:245-255, 1997), the Arabidopsis thaliana SUC2 sucrose-H⁺ symporter promoter (Truernit et al., Planta 196:564-570, 1995), and thylakoid membrane protein promoters from spinach (psaD, psaF, psaE, PC, FNR, atpC, atpD, cab, and rbcS). Additional exemplary promoters that can be used to drive gene transcription in stems, leafs, and green tissue are described in U.S. Patent Application Publication No. 2007/0006346, herein incorporated by reference in its entirety. Additional promoters that result in preferential expression in plant green tissues include those from genes such as Arabidopsis thaliana ribulose-1,5-bisphosphate carboxylase (Rubisco) small subunit (Fischhoff et al., Plant Mol. Biol. 20:81-93, 1992), aldolase and pyruvate orthophosphate dikinase (PPDK) (Taniguchi et al., Plant Cell Physiol. 41(1):42-48, 2000).

In some embodiments, the promoters may be altered to contain one or more enhancers to assist in elevating gene expression. Examples of enhancers that can be used to promote gene expression are known in the art. Enhancers are often are found 5′ to the start of transcription in a promoter that functions in eukaryotic cells, but can often be inserted upstream (5′) or downstream (3′) to the coding sequence. In some instances, these 5′ enhancing elements are introns. Non-limiting examples of enhancers include the 5′ introns of the rice actin 1 and rice actin 2 genes (see, U.S. Pat. No. 5,641,876), the maize alcohol dehydrogenase gene intron, the maize heat shock protein 70 gene intron (U.S. Pat. No. 5,593,874), and the maize shrunken 1 gene intron.

In some embodiments, the DNA construct or vector can also contain a non-translated leader sequence derived from a virus. Non-limiting examples of non-translated leader sequences that can promote transcription include those from Tobacco Mosaic Virus (TMV, the “W-sequence”), Maize Chlorotic Mottle Virus (MCMV), and Alfalfa Mosaic Virus (AMV) (see, e.g. Gallie et al., Nucl. Acids Res. 15: 8693-8711, 1987; Skuzeski et al., Plant Mol. Biol. 15: 65-79, 1990). Additional exemplary leader sequences include: picornavirus leaders, for example, EMCV leader (Encephalomyocarditis 5′ noncoding region) (Elroy-Stein et al., Proc. Natl. Acad. Sci. U.S.A. 86:6126-6130, 1989); potyvirus leaders, for example, TEV leader (Tobacco Etch Virus); MDMV leader (Maize Dwarf Mosaic Virus); human immunoglobulin heavy-chain binding protein (BiP) leader (Macejak et al., Nature 353: 90-94, 1991; untranslated leader from the coat protein mRNA of alfalfa mosaic virus (AMV RNA 4) (Jobling et al., Nature 325:622-625, 1987); tobacco mosaic virus leader (TMV) (Gallie et al., Mol. Biol. RNA, pages 237-256, 1989); and Maize Chlorotic Mottle Virus leader (MCMV) (Lommel et al., Virology 81:382-385, 1991). See also, Della-Cioppa et al., Plant Physiology 84:965-968, 1987.

In some embodiments, the DNA constructs or vectors can also contain a 3′ element that may contain a polyadenylation signal and/or site. Well-known 3′ elements include those from Agrobacterium tumefaciens genes, such as nos 3′, tml 3′, tmr 3′, tins 3′, ocs 3′, tr7 3′, see, e.g., the 3′ elements described in U.S. Pat. No. 6,090,627, incorporated herein by reference. The 3′ elements can also be derived from plant genes, e.g., the 3′ elements from a wheat (Triticum aesevitum) heat shock protein 17 (Hsp17 3′), a wheat ubiquitin gene, a wheat fructose-1,6-biphosphatase gene, a rice glutelin gene, a rice lactate dehydrogenase gene, and a rice beta-tubulin gene, all of which are described in U.S. Patent Application Publication No. 2002/0192813 (herein incorporated by reference), the pea (Pisum sativum) ribulose biphosphate carboxylase gene (rbs 3′), and the 3′ elements from the genes within the host plant. In some embodiments, the 3′ element can also contain an appropriate transcriptional terminator, such as a CAMV 35S terminator, the tml terminator, the nopaline synthase terminator, and the pea rbcs E9 terminator.

In some embodiments, the DNA constructs or vectors include an inducible promoter. Inducible promoters drive transcription in response to external stimuli, such as chemical agents or environmental stimuli. For example, inducible promoters can confer transcription in response to hormones, such as gibberellic acid or ethylene, or in response to light or drought. Non-limiting examples of inducible promoters are described in Guo et al., Plant J. 34:383-392, 2003, and Chen et al., Plant J. 36:731-40, 2003.

In some embodiments, the DNA constructs and vectors can also include a nucleic acid encoding a transit peptide or signaling peptide for the targeting of an exogenous chromophore to a plastid, e.g., a chloroplast. For example, the targeting of an exogenous chromophore to the chloroplast can be controlled by a signal sequence found at the amino terminal end of an exogenous chromophore, which is cleaved during chloroplast import (e.g. Comai et al., J. Biol. Chem. 263:15104-15109, 1988). Exemplary signal sequences can be fused to a heterologous gene product (e.g., an exogenous chromophore) to affect the import of a heterologous product (e.g., an exogenous chromophore) into a chloroplast (see, e.g., van den Broeck et al., Nature 313: 358-363, 1985). DNA encoding for appropriate signal sequences can be isolated from the 5′ end of the cDNAs encoding the RUBISCO protein, the CAB protein, the EPSP synthase enzyme, the GS2 protein, and many other proteins which are known to be chloroplast localized. See, for example, the section entitled “Expression With Chloroplast Targeting” in Example 37 of U.S. Pat. No. 5,639,949 (herein incorporated by reference).

Non-limiting examples of transit or signal peptides that can be used include: the plastidic Ferredoxin:NADP⁺ oxidoreductase (FNR) of spinach, which is described in Jansen et al., Current Genetics 13:517-522, 1988. In particular, the sequence ranging from the nucleotides −171 to 165 of the cDNA sequence in Jansen et al., Current Genetics 13:517-522, 1998 can be used, which comprises the 5′ non-translated region, as well as the sequence encoding the transit peptide. Another example is the transit peptide of the waxy protein of maize including the first 34 amino acid residues of the mature waxy protein (Klosgen et al., Mol. Gen. Genet. 217:155-161, 1989). It is also possible to use this transit peptide without the first 34 amino acids of the mature protein. Furthermore, the signal peptides of the ribulose bisposphate carboxylase small subunit (Wolter et al., Proc. Natl. Acad. Sci. U.S.A. 85:846-850, 1988; Nawrath et al., Proc. Natl. Acad. Sci. U.S.A. 91:12760-12764, 1994), the NADP malate dehydrogenase (Galiardo et al., Planta 197:324-332, 1995), the glutathione reductase (Creissen et al., Plant J. 8: 167-175, 1995) or the R1 protein Lorberth et al. (Nature Biotechnology 16:473-477, 1998) can be used.

Additional thylakoid-targeting and stromal-targeting signal peptides are described in Fan et al., Biochem. Biophys. Res. Comm. 398:438-443, 2010; Jarvis et al., Curr. Biol. 14:R1064-1077, 2004; McFadden, J. Eukaryot. Microbiol. 46:339-346, 1999; Robinson et al., Plant Mol. Biol. 38:209-221, 1998; Brink et al., J. Biol. Chem. 270:20808-20815, 1995; and Von Heijne et al., Eur. J. Biochem. 180:535-545, 1989.

Additional examples of chloroplast transit peptides are described in U.S. Pat. No. 5,188,642 and U.S. Pat. No. 5,728,925, incorporated herein by reference. Another example of transit peptide is the transit peptide of the Arabidopsis EPSPS gene, see, e.g., Klee, H. J. et al. (MGG 210:437-442, 1987).

In some embodiments, the DNA construct or vector can also include a selectable marker gene to allow for selection of stable transformants (see, e.g., the selectable markers described herein). In some embodiments, the chromophore can be used as a marker gene to select stable transformants (e.g., by measuring the specific wavelength of light emitted by the chromophore).

An exemplary DNA vector that can be used is a pZS 197 vector. This vector contains a chimeric aadA gene under the control of the ribosomal RNA operon promoter (Prrn) and the 3′ region of the plastid psbA gene (Prrn/aadA/TpsbA) and contains the plastid rbcL and accD genes for targeting to the large single copy region of chloroplast genome. Another exemplary DNA vector that can be used is the pMON30125 inverted repeat vector, which is a derivative of pPRV111A. The pMON30125 vector contains a chimeric aadA gene driven by the PpsbA and TpsbA expression signals. Additional exemplary DNA vectors and constructs that can be used to express an exogenous chromophore are known in the art.

Methods of Transformation

Transformation techniques for plants are well known in the art and include Agrobacterium-based techniques (see, e.g., U.S. Pat. Nos. 5,635,055; 5,824,877; 5,591,616; 5,981,840; and 6,384,301) and techniques that do not require Agrobacterium. Non-Agrobacterium techniques involve the uptake of exogenous genetic material directly by protoplasts or cells. This can be accomplished by polyethylene glycol (PEG)- or electroporation-mediated uptake (see, e.g., U.S. Pat. No. 5,384,253), particle bombardment-mediated delivery (see, e.g., U.S. Pat. Nos. 5,015,580; 5,550,318; 5,538,880; 6,160,208; 6,399,861; and 6,403,865), protoplast transformation (see, e.g., U.S. Pat. No. 5,508,184) or microinjection. Non-limiting examples of these techniques are described by Paszkowski et al., EMBO J. 3:2717-2722, 1984; Potrykus et al., Mol. Gen. Genet. 199:169-177, 1985; Reich et al., Biotechnology 4:1001-1004, 1986; and Klein et al., Nature 327:70-73, 1987.

Transformation using Agrobacterium has also been described (see, e.g., WO 94/00977 and U.S. Pat. No. 5,591,616, each of which is incorporated herein by reference). In each case, the transformed cells are regenerated to whole plants using standard techniques known in the art. Many vectors are available for transformation using Agrobacterium tumefaciens. These vectors typically carry at least one T-DNA border sequence and include vectors such as pBIN19 (Bevan, Nucl. Acids Res. 11:369, 1984). The binary vector pCIB10 contains a gene encoding kanamycin resistance for selection in plants and T-DNA right and left border sequences and incorporates sequences from the wide host-range plasmid pRK252 allowing it to replicate in both E. coli and Agrobacterium (Rothstein et al., Gene 53:153-161, 1987). Transformation of the target plant species by recombinant Agrobacterium usually involves co-cultivation of the Agrobacterium with explants from the plant and follows protocols well known in the art. The transformed tissue is regenerated on selectable medium carrying the antibiotic or herbicide resistance marker present between the binary plasmid T-DNA borders.

Another approach to transforming a plant cell with a gene involves propelling inert or biologically active particles at plant tissues and cells. This technique is disclosed in U.S. Pat. Nos. 4,945,050; 5,036,006; and 5,100,792 (each of which is incorporated herein by reference). Generally, this procedure involves propelling inert or biologically active particles at the cells under conditions effective to penetrate the outer surface of the cell. Gordon-Kamm et al., Plant Cell 2:603-618, 1990; Fromm et al., Biotechnology 8:833-839, 1990; WO 93/07278; and Koziel et al., Biotechnology 11:194-200, 1993 describe exemplary methods of particle bombardment to achieve transformation of plant cells. Exemplary methods of transforming plastids using particle bombardment are described in Svab et al., Proc. Natl. Acad. Sci. U.S.A. 90:913-917, 1993; Svab et al., Proc. Natl. Acad. Sci. U.S.A. 87:8526-8530, 1990; McBride et al., Proc. Natl. Acad. Sci. U.S.A. 91:7301-7305, 1994; Day et al., Plant Biotech. J. 9:540-553, 2011.

As noted above, plant cells can also be transformed using PEG or electroporation. Non-limiting examples of techniques that utilize PEG or electroporation to transform plant cells are described in EP 0292435, EP 0392225, and WO 93/07278.

Plastid transformation can be also be used to produce transgenic plants expressing a heterologous chromophore without the need for nuclear genome transformation. Plastid transformation technology is extensively described in U.S. Pat. Nos. 5,451,513; 5,545,817; and 5,545,818 (each of which is herein incorporated by reference) and in WO 95/16783, (incorporated by reference in its entirety); and in McBride et al., Proc. Natl. Acad. Sci. U.S.A. 91: 7301-7305, 1994; and Okumura et al., Transgenic Res. 15:637-646, 2006. The basic technique for chloroplast transformation involves introducing regions of cloned plastid DNA flanking a selectable marker together with the gene of interest into a suitable target tissue, e.g., using biolistics or protoplast transformation (e.g., calcium chloride- or PEG-mediated transformation). The 1 to 1.5 kb flanking regions, termed targeting sequences, facilitate homologous recombination with the plastid genome, and allow for the replacement or modification of specific regions of the plastid DNA. Initially, point mutations in the chloroplast 16S rRNA and rps12 genes conferring resistance to spectinomycin and/or streptomycin were utilized as selectable markers for transformation (see, e.g., Svab et al., Proc. Natl. Acad. Sci. U.S.A. 87:8526-8530, 1990; Staub et al., Plant Cell 4, 39-45, 1992). This achieved stable homoplasmic transformants at a frequency of approximately one per 100 bombardments of target leaves. The presence of cloning sites between these markers allowed creation of a plastid targeting vector for introduction of foreign genes (Staub et al., EMBO J. 12, 601-606, 1993). Substantial increases in transformation frequency were obtained by replacement of the recessive rRNA or tau-protein antibiotic resistance genes with a dominant selectable marker, the bacterial aadA gene encoding the spectinomycin-detoxifying enzyme aminoglycoside-3′-adenyltransferase (Svab et al., Proc. Natl. Acad. Sci. U.S.A. 90:913-917, 1993). Other selectable markers useful for plastid transformation are known in the art. Another example of a vector that can be used for plastid (e.g., chloroplast transformation) is vector pPH143 (WO 97/32011). Plastid transformation, in which genes are inserted by homologous recombination into all of the several thousand copies of the circular plastid genome present in each plant cell, takes advantage of the enormous copy number of plastid DNA over nuclear-expressed genes to permit expression levels that can readily exceed 10% of the total soluble plant protein.

Transient transformation can also be used to express a heterologous chromophore in plant cell or plant. Non-limiting examples of transient transformation of plant tissues include leaf infiltration, vacuum infiltration, infection with Agrobacterium, or bombardment of target tissues with DNA-coated particles.

Assays for Measuring Photosynthetic Activity

The amount of photosynthesis performed in a plant cell or plant can be indirectly detected by measuring the amount of starch produced by the transgenic plant or plant cell. The amount of photosynthesis in a plant cell culture or a plant can also be detected using a CO₂ detector (e.g., a decrease or consumption of CO₂ indicates an increased level of photosynthesis) or a O₂ detector (e.g., an increase in the levels of O₂ indicates an increased level of photosynthesis (see, e.g., the methods described in Silva et al., Aquatic Biology 7:127-141, 2009; and Bai et al., Biotechnol. Lett. 33:1675-1681, 2011). Photosynthesis can also be measured using radioactively labeled CO₂ (e.g., ¹⁴CO₂ and H¹⁴CO₃⁻) (see, e.g., the methods described in Silva et al., Aquatic Biology 7:127-141, 2009, and the references cited therein). Photosynthesis can also be measured by detecting the chlorophyll fluorescence (e.g., Silva et al., Aquatic Biology 7:127-141, 2009, and the references cited therein). Additional methods for detecting photosynthesis in a plant are described in Zhang et al., Mol. Biol. Rep. 38:4369-4379, 2011).

Plant Reagents and Experimental Methods

The products and processes described herein may be constructed from or carried out using reagents and methods know in the art. Such reagents and methods include those for plant gene and protein expression systems, including systems that provide for expression in the cytoplasm and specific compartments of the chloroplast. Plant transformation systems are also known in the art. Examples include transformation utilizing agrobacterium and ballistic projectiles. Transformation may be to the nucleus or chloroplast, e.g., the chloroplast thylakoid or stroma and may be either a stable or transient transformation (e.g., using the exemplary methods described herein). Methods of assaying photosynthetic activity are also known in the art.

Plants

In some embodiments, the transgenic plant is a monocot or a dicot. Examples of monocot transgenic plants include, e.g., a meadow grass (blue grass, Poa), a forage grass (e.g., festuca and lolium), a temperate grass (e.g., Agrostis), and cereals (e.g., wheat, oats, rye, barley, rice, sorghum, and maize). Examples of dicot transgenic plants include, e.g., tobacco, legumes (e.g., lupins, potato, sugar beet, pea, bean, and soybean), and cruciferous plants (family Brassicaceae) (e.g., cauliflower and rape seed). Thus, the transgenic plants provided herein include a broad range of plants, including, but not limited to, species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Pannisetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna, and Zea.

In some embodiments, the transgenic plant is a tree or shrub (e.g., a eucalyptus tree or shrub). Non-limiting examples of eucalyptus include, without limitation, the following species and crosses thereof: E. botryoides, E. bridgesiana, E. camaldulensis, E. cinerea, E. globule, E. grandis, E. gunii, E. nicholii, E. pulverulenta, E. robusta, E. rudis, E. saligna, E. Tereticornis, E. Urophilla, E. viminalis, E. dunnii and a cross hybrids of any of the preceding species especially Eucalyptus grandis and Eucalyptus urophylla. Other examples include Poplar species, e.g., P. deltoides, P. tremula, P. alba, P. nigra (euramericana), P. nigra (canadensis), P. tremula, P. trichocarpa, P. rouleauiana, P. balsamifera, P. maximowiczii and crosses thereof; and Pine species (Genus=Pinus).

In some embodiments, the transgenic plant is an ornamental plant.

Methods of Use

Plants normally compete for sunlight. Held upright by stems and trunks, leaves configure a canopy that absorbs light and influences photosynthetic rates and growth beneath them. Leaves that are shaded by other leaves have much lower photosynthetic rates. Densely grown plants such as forestry trees need to compete for light more than less densely grown plants. The transgenic plants of the current invention have enhanced photosynthesis by enabling the capture of more photons from non utilized light wavelengths that can then be absorbed to generate energy for increased photosynthetic rates compared to wild type plants, thus increasing biomass accumulation and growth.

In one aspect, photosynthetic activity of plants cultivated in a greenhouse may be enhanced by matching chromophore excitation/emission spectra with the wavelength emissions from greenhouse lights, e.g., especially LED or other energy efficient light sources. Plants may be grown, for example, a greenhouse is equipped with LEDs or some other light source or several light sources including broad range sources bolstered by specific range or ranges that emits light which is adapted to be optimal for the specific endogenous chromophores that are expressed in the plant.

Also provided are methods of increasing the nitrogen content of soil that include planting (cultivating) a transgenic nitrogen-fixing plant expressing at least one of the chromophores described herein. In some embodiments, the transgenic nitrogen-fixing plant can be cultivated in proximity (e.g., in every other row or in every second, third, fourth, fifth, sixth, seventh, eighth, ninth, or tenth row) to a different plant (e.g., a non-transgenic plant or a different transgenic plant as described herein). In some embodiments, the method can include the step of plowing (tilling) the transgenic nitrogen-fixing plant into the soil and allowing for the decomposition of the transgenic nitrogen-fixing plant tissue in the soil. In some embodiments, several growth cycles can be done during the year.

Examples of nitrogen-fixing plants include legumes. Other examples of nitrogen-fixing plants include limited numbers of species of Parasponia, Actinorhizal (e.g., alder and bayberry), Rosaceae (orders Cucurbitales, Fagales, and Rosales). Preferred nitrogen-fixing organisms are legumes. Examples of legumes include, without limitation, tropical legumes of the genera Glycine (soybean), Phaseolus (common bean), Lotus, and Vigna and temperature legumes, Pisum (pea), Medicago (alfalfa), Trifolium (clover), and Vicia (vetch).

### EXAMPLES

### Example 1

### Construct Preparation and Plant Transformation

One or more constructs comprising chromophore(s) that singly or together absorb light and emit light that may be utilized to drive photosynthesis are constructed and transformed into plants and such transformants are isolated.

### Example 2

### Characterization of Fluorescence in Transgenic Plant Leaves

Fluorescence absorption and emission of recombinant proteins are measured using fluorescent microscopy. Fresh leaves from plants transformed as set out in Example 1 and untransformed controls are examined under appropriate light excitation wavelengths or wavelengths, i.e., the excitation maxima the tested transgenic chromophore or chromophores to be examined, using compound fluorescence microscope, e.g., Zeiss III-RS or Zeiss Axiovert 100S (Zeiss). Images are recorded electronically or on film.

### Example 3

### Photosynthetic Activity in Transgenic Plants

Chlorophyll Fluorescence Measurements

Measurements of modulated chlorophyll fluorescence emission from the upper surface of leaves are made using a pulse amplitude modulation fluorometer (PAM-101; H. Waltz, Effeltrich, Germany (MONOTORING-PAM Chlorophyll Fluorometer). Each measuring head generates modulated fluorescence excitation light, continuous actinic light and saturation flashes by a blue power LED. Light sources and signal detection and saturating light are held 5 mm from the upper surface of the leaves. Fiber optics is used to guide light from the power and control unit to the sample, and to direct light from the sample back. The intensity of the measuring, modulated red light is ˜0.1 μmol·m⁻²s⁻¹. Leaves are dark-adapted in a zero-light environment for 10 min before measuring the induction of fluorescence. The measuring beam [excitation beam] is used to induce the minimum fluorescence (F0). Saturating flashes are provided to completely reduce the PSII acceptor site QA and to measure the maximum fluorescence yield (Fm). The intensity of the saturating light flash (1 s) used for the measurements of Fm is 3000 μmol·m⁻²s⁻¹. Variable fluorescence (Fv) is calculated as Fm−F0. The ratio Fv:Fm reflects the potential yield of the photochemical reaction of PSII (Krause and Weis, Annu. Rev. Plant Physiol. Plant Mol. Biol. 42:313-349, 1991).

Gas Exchange

Gas exchange measurements are performed using a GFS-3000 Portable Gas Exchange Fluorescence System (Walz, http://www.walz.com). Water and CO₂ concentrations at the inlet and outlet of the cuvette are measured using a differential infrared gas analyzer (IRGA). Cuvette flow is adjusted to 750 μmol s⁻¹, and its area is 3 cm². Plant leaves are light adapted at a saturating PFD of 1000 and 400 μmol mol⁻¹CO₂ (Ca) and light response curves are recorded at nine different light intensities (0-1000 μmol m⁻² s⁻¹) by decreasing the applied PFD in a stepwise fashion. CO₂ response curves are obtained by measuring the net photosynthesis rate depending on varying CO₂ concentrations in the cuvette. Leaves are adjusted to 750 μmol m⁻² s⁻¹ PFD and 400 μmol mol⁻¹ CO₂. Measurements are started after leaves show a constant photosynthetic rate. CO₂ concentration is reduced stepwise to a Ca of 50 μmol mol⁻¹ CO₂, followed by 400 μmol mol⁻¹ CO₂, to regain initial CO₂ assimilation rates. Ca is subsequently increased stepwise to 1000 μmol mol⁻¹.

### Example 4

### Determination of Rubisco and Chlorophyll Content and Morphological Characteristics

Rubisco content is determined by extracting soluble proteins from leaf samples and performing western blot analysis using Rubisco-LSU antibody (Agrisera, www.agrisera.com) as described by Uehlein et al., Plant Cell 20:648-657, 2008). Protein content was quantified with Quantity One® (Bio-Rad, http://www.bio-rad.com). Chlorophyll is extracted from leaf samples and determined as described using acetone as the solvent (Porra et al., Biochimica et Biophysica Acta, 975:384-394, 1989). Leaf anatomical parameters are examined of 6-week-old plants. Leaf number is counted and the stem diameter is measured at three different points per plant. Leaf area is determined after scanning with IMAGEJ. Stomata length and density are assessed by making imprints of the leaf abaxial side with clear nail polish. After an incubation of 3-5 min at 20° C., light microscope pictures are taken and analyzed with IMAGEJ.

### Example 5

### Plant Growth

Transgenic plants expressing exogenous chromophores and control plants are grown under the following conditions:

(i) In the growth room under green LEDs (530 nm) and yellow LEDs (580 nm);

(ii) In the net house under different shade rate (20%-70%);

(iii) In the field under regular planting density (3×3 meters) and high density (3×1 meters) conditions.

Bio mass accumulations of transgenic and control plants are measured and compared according to standard techniques.

### Example 6

### Eucalyptus Transformed with an Exogenous Chromophore Demonstrate Increased Growth

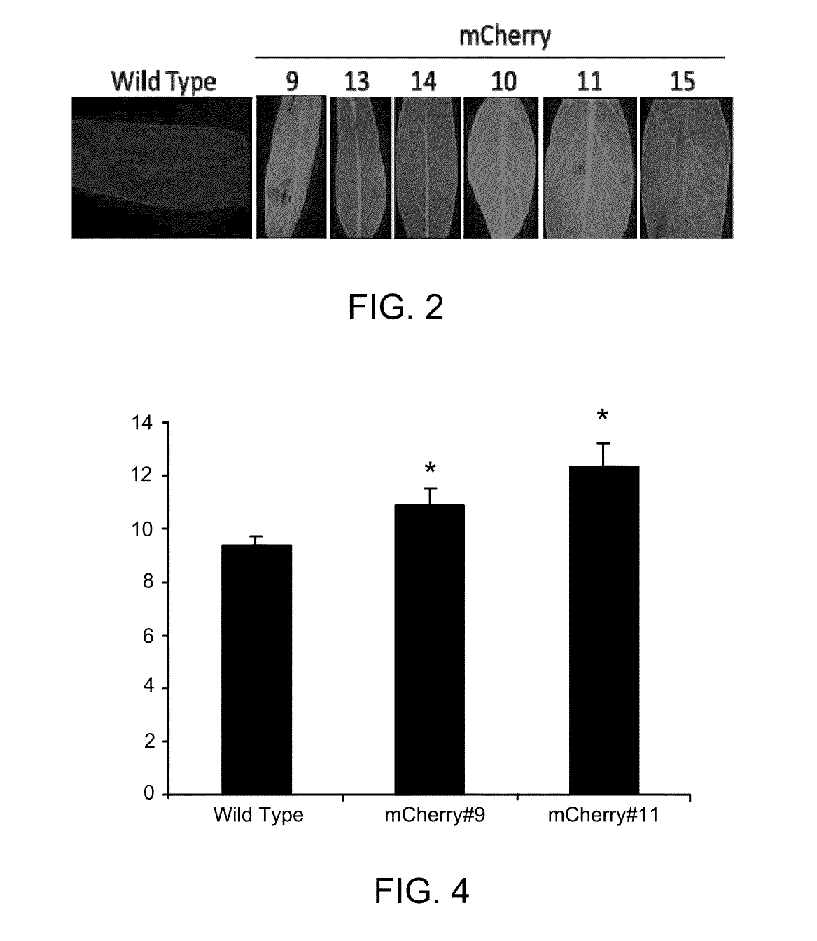

Experiments were performed using eucalyptus in order to confirm that transformation of a plant with a chromophore would result in increased photosynthesis and plant growth. In these experiments, the synthetic gene of mCherry, SEQ ID NO: 16 (GenBank: AAV52164.1 with a G230S mutation) was cloned into a plasmid pBI121 (GenBank: AF485783.1) under the CaMV 35S promoter and with the NOS terminator using XbaI and SacI restriction sites. Agrobacterium EAH105 was electrotransformed, selected for 48 hours on kanamycin plates (100 μg/ml), and used for plant transformation. Eucalyptus transformation using a protocol essentially as described in Prakash et al., In Vitro Cell Dev Biol.-Plant 45:429-434, 2009. Briefly, shoots of Eucalyptus were propagated in vitro on Murashige and Skoog (MS) basal salt medium consisting of 3% (w/v) sucrose and 0.8% (w/v) agar. Transgenic plant selection was performed using kanamycin and by detection of mCherry fluorescence in whole single shoots in the selection plates by standard protocols. Red fluorescence was detected using Olympus SZX2-ZB16 zoom fluorescence stereoscope with a SZX2-FRFP1 Fluorescence filter set (Exciter filter BP530-550 barrier filter BA575IF). The positive plants were rooted and propagated by standard protocols and later were tested for fluorescence intensity. One detached leaf (0.5 cm in size) from the middle of each transgenic shoot was tested for fluorescence intensity under the fluorescence stereoscope. Fluorescence score was in arbitrary units on a scale from 1-5 as seen by the eye (FIG. 2).

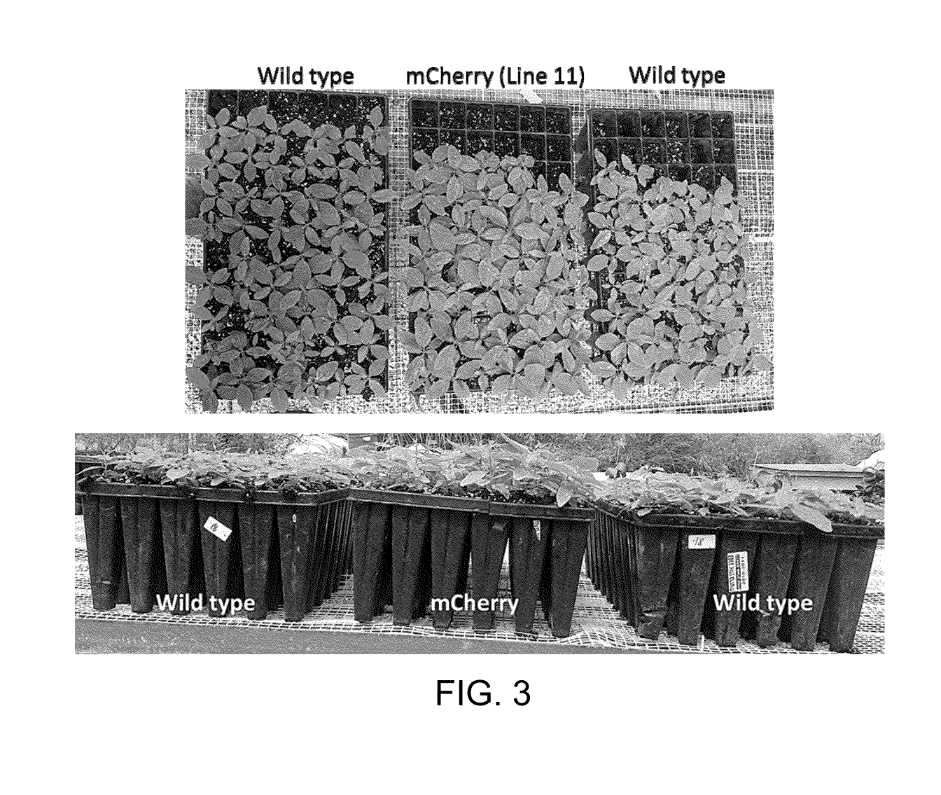

Selected plants performing at different fluorescence intensities were transferred to the greenhouse (24° C., 14 hours natural sunlight). The transgenic plants were grown in the greenhouse for 36 days and measured for height (from the bottom of the stem to the top). The transgenic plants with significant expression of mCherry show increased growth as compared to wild type control plants (see, FIGS. 3 and 4). These data indicate that transgenic plants expressing a chromophore have increased photosynthesis that results in increased plant growth.

A number of embodiments of the invention have been described. Nevertheless, it will be understood that various modifications may be made without departing from the spirit and scope of the invention. Accordingly, other embodiments are within the scope of the following claims. Other features, objects, and advantages of the invention will be apparent from the description and drawings, and from the claims.

Each patent and non-patent literature reference cited herein is hereby incorporated by reference in its entirety.

What is claimed:

1. A transgenic plant with improved photosynthetic activity comprising an exogenous first chromophore that absorbs a first wavelength of light in a range that is suboptimal for photosynthetic activity in said plant and which upon absorbing said first wavelength of light said chromophore emits a second wavelength of light in a range that is effective for said photosynthetic activity in said plant.

2. The transgenic plant according to claim 1 further comprising a second exogenous chromophore that absorbs a third wavelength of light in a range that is suboptimal for photosynthetic activity in said plant and upon absorbing the third wavelength of light emits said first wavelength of light.

3. The transgenic plant according to claim 2, wherein the second exogenous chromophore has a maximum emission wavelength that is identical or near the maximum absorption wavelength of the first chromophore.

4. The transgenic plant according to claim 1 wherein said second wavelength of light is efficiently absorbed by a chlorophyll or tetreterpenoid compound.

5. The transgenic plant according to claim 4 wherein said second wavelength of light is efficiently absorbed by a chlorophyll a or chlorophyll b.

6. The transgenic plant according to claim 2 wherein transfer of energy between said chromophores occurs by means of resonance energy transfer (RET).

7. The transgenic plant according to claim 1 wherein said exogenous chromophore is localized to the chloroplast of said plant.

8. The transgenic plant according to claim 7 wherein said exogenous chromophore is localized to the thylakoid membrane of said plant.

9. The transgenic plant according to claim 2 comprising the exogenous chromophores turboYFP, mKO1, DsRed-Express2 and Turbo FP650.

10. The transgenic plant according to claim 1 wherein said transgenic plant is a legume or a eucalyptus species.

11. The transgenic plant according to claim 10 wherein said transgenic plant is a legume.

12. A method of co-cultivating plants comprising co-cultivating the transgenic plant according to claim 1 with a second plant under conditions wherein said transgenic plant is shaded by said second plant.

13. The method of claim 12 wherein said second plant is a eucalyptus or sugar cane plant and said transgenic plant is a legume.

14. A method of co-cultivating plants comprising co-cultivating the transgenic plant according to claim 2 with a second plant under conditions wherein said transgenic plant is shaded by said second plant.

15. The method of claim 14 wherein said second plant is a eucalyptus or sugar cane plant and said transgenic plant is a legume.

16. A method of growing a plant under shaded conditions comprising growing the transgenic plant according to claim 1 under shaded conditions.

17. A method of growing a plant under shaded conditions comprising growing the transgenic plant according to claim 2 under shaded conditions.

18. A method of increasing the nitrogen level in soil, the method comprising planting a transgenic plant according to claim 1, wherein the transgenic plant is a legume.

19. A method of increasing the nitrogen level in soil, the method comprising planting a transgenic plant according to claim 2, wherein the transgenic plant is a legume.
